(19) United States
(12) Patent Application Publication
(10) Pub. No.: US 2026/0194434 A1
(43) Pub. Date: Jul. 9, 2026

(54) TRANSDERMAL MIGRATION DEVICE

(71) Applicant: Jinan University, Guangzhou (CN)

(72) Inventors: Chenchou WU, Guangzhou (CN); Chunyan CHEN, Guangzhou (CN); Yun HONG, Guangzhou (CN); Yongping ZENG, Guangzhou (CN)

(21) Appl. No.: 19/433,431

(22) Filed: Dec. 26, 2025

(30) Foreign Application Priority Data
Jan. 9, 2025 (CN) 202510032859.9

(51) Int. Cl. G01N 13/00; G01N 33/15

(52) U.S. Cl. CPC G01N 13/00; G01N 33/15; G01N 2013/003

(57) ABSTRACT

The present invention relates to a transdermal migration device, including a receptor cell for holding human body fluid, an adsorbent phase, a connecting cap, a diffusion membrane, sealing components, a donor cell for placing a sample, and a top cover. the adsorbent phase is placed inside the receptor cell; the connecting cap has a shape of a groove and is hollow in the middle, with upper and lower ends detachably connected to the donor cell and the receptor cell, respectively; the diffusion membrane is laid flat in the groove of the connecting cap and fastened by the donor cell, with upper and lower surfaces of the membrane tightly contacting the sample in the donor cell and the human body fluid in the receptor cell, respectively; the connecting cap is provided with vent holes to communicate the receptor cell with ambient air.

### CROSS-REFERENCE TO RELATED APPLICATIONS

The present application claims priority to Chinese patent application No. 202510032859.9, titled “TRANSDERMAL MIGRATION DEVICE”, filed on 9 January 2025, the entire contents of which are incorporated herein by reference.

### FIELD OF TECHNOLOGY

The present invention relates to the technical field of risk assessment of human transdermal exposure to consumer products, and in particular to a transdermal migration device.

### BACKGROUND

The skin, as the largest organ of a human body, is constantly exposed to environments and numerous chemicals. Contact between the skin and a surface of a consumer product is a common scenario for skin exposure. Chemicals in the consumer product can enter the human body through transdermal exposure from the surface of the consumer product. Currently, a level of skin exposure to the chemicals from the consumer products can be estimated using methods such as surface wiping or artificial sweat immersion, to investigate sweat-mediated migration behavior of these chemicals and assess a human transdermal exposure risk. The surface wiping is influenced by a wiping force and wiping liquid, and can only evaluate a release potential within an extremely short period. In the artificial sweat immersion method, the consumer product is usually cut into small pieces or completely crushed for immersion. This method can effectively increase a contact area between the consumer product and sweat, and shorten assessment duration. However, this method greatly damages a structure of the consumer product and ignores an actual scenario where the skin only contacts the surface of the consumer product.

The Franz diffusion cell is commonly used in transdermal tests for formulations such as patches, suppositories, and semi-solid preparations, and can objectively reflect a transdermal release process of the formulation. However, this device is mainly used for polar and highly soluble chemicals, whereas chemicals with low solubility often fall below a detection limit of an instrument. Current studies mostly employ the addition of an adsorbent phase, such as XAD resin, polydimethylsiloxane, or polyethylene, to enrich low-solubility chemicals in environmental water samples, thereby increasing a detection amount of a target substance. However, repeated replacement of an adsorbent greatly affects a position of a diffusion membrane in the Franz diffusion cell. In addition, the chemicals in the consumer product include nonpolar and low-solubility trace substances, which are easily affected by potential environmental background contamination.

### SUMMARY

In view of the problems in the prior art, the present invention is intended to provide a transdermal migration device that is easy to operate, resistant to environmental background interference, and capable of highly simulating a migration process of a chemical from a surface of a consumer product through the skin into human body fluid.

To achieve the above objective, the following technical solutions are used in the present invention:

a transdermal migration device, including a receptor cell for holding human body fluid, an adsorbent phase, a connecting cap, a diffusion membrane, sealing components, a donor cell for placing a sample, and a top cover, where

the adsorbent phase is placed inside the receptor cell;

the connecting cap has a shape of a groove and is hollow in the middle, with upper and lower ends detachably connected to the donor cell and the receptor cell, respectively;

the diffusion membrane is laid flat in the groove of the connecting cap and fastened by the donor cell, with upper and lower surfaces of the membrane tightly contacting the sample in the donor cell and the human body fluid in the receptor cell, respectively;

the connecting cap is provided with vent holes to communicate the receptor cell with ambient air, and the sealing components are detachably plugged into the vent holes; and

the top cover covers the top of the donor cell.

Further, the connecting cap is threadedly connected to both the donor cell and the receptor cell, and the diffusion membrane is fastened by threaded engagement between the connecting cap and the donor cell.

Further, a lower circumferential surface of the connecting cap is provided with an external thread, and an upper circumferential surface of the receptor cell is provided with a matching internal thread; and an upper circumferential surface of the connecting cap is provided with an internal thread, and a lower circumferential surface of the donor cell is provided with a matching external thread.

Further, a small sealing ring is provided on the diffusion membrane, and the donor cell is pressed tightly against the small sealing ring.

Further, a large sealing ring is provided at a connection between the connecting cap and the receptor cell.

Further, an annular groove is provided on the lower circumferential surface of the connecting cap, and the large sealing ring is fitted in the annular groove.

Further, the vent holes are screw holes, the sealing components are screws, and the screws are threadedly connected to the screw holes.

Further, the adsorbent phase is made of XAD resin, polydimethylsiloxane, or polyethylene.

Further, the diffusion membrane is made of dialysis membrane or skin membrane.

Further, the receptor cell, the connecting cap, the donor cell, and the top cover are made of stainless steel, glass, or plastic.

In summary, the present invention has the following advantages.

The transdermal migration device in the present invention is designed to highly simulate the migration process of the chemical from the surface of the consumer product through the skin into the human body fluid.

(1) The diffusion membrane is placed in the groove of the connecting cap and fastened by threaded engagement between the connecting cap and the donor cell. The upper and lower surfaces of the diffusion membrane tightly contact the sample in the donor cell and the human body fluid in the receptor cell, respectively, enabling the sample to contact the human body fluid only through the surface of the diffusion membrane, thereby implementing unidirectional migration of the chemical from the sample into the human body fluid.

(2) The receptor cell, the connecting cap, and the donor cell can be assembled by threaded engagement, where only the receptor cell and the connecting cap need to be unscrewed to disassemble the device, without moving the diffusion membrane, which facilitates recovery and replacement of the adsorbent phase.

(3) The vent holes in the connecting cap allow for easy discharge of gas from the receptor cell and extraction of the human body fluid from the receptor cell, and can be sealed by the sealing components to block the vent holes, thereby cutting off the connection between the receptor cell and external atmosphere and ensuring airtightness of the device.

(4) After assembly, the device is fully enclosed, preventing reduction of the human body fluid in the receptor cell due to leakage or evaporation, resisting environmental background interference, and improving the accuracy of detection and analysis.

### BRIEF DESCRIPTION OF THE DRAWINGS

FIG. 1 shows a schematic structural diagram of a transdermal migration device according to the present invention.

### DESCRIPTION OF THE EMBODIMENTS

The present invention will be further described in detail below.

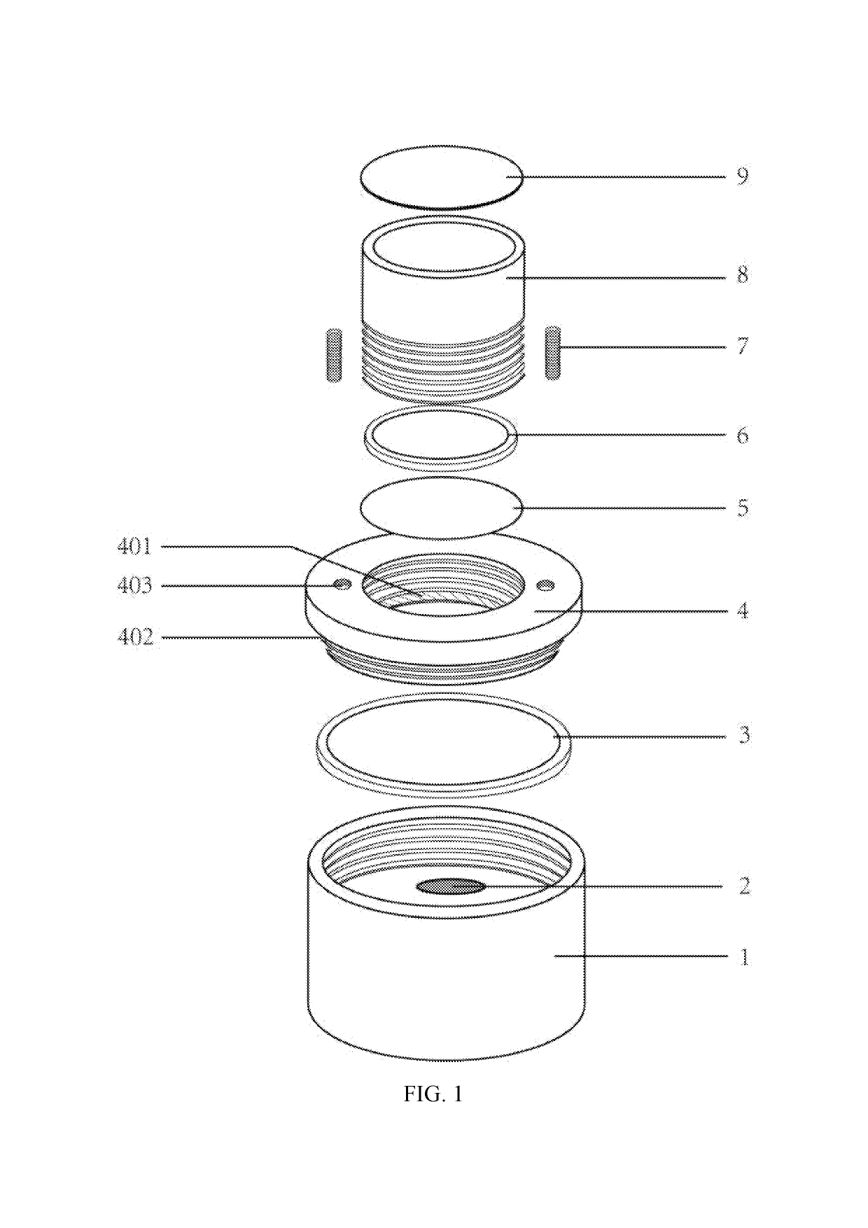

FIG. 1 shows a transdermal migration device, including a receptor cell 1, an adsorbent phase 2, a large sealing ring 3, a connecting cap 4, a diffusion membrane 5, a small sealing ring 6, screws 7, a donor cell 8, and a top cover 9.

The receptor cell 1 is configured to hold human body fluid, the connecting cap 4 is configured to connect the receptor cell 1 and the donor cell 8, and the donor cell 8 is configured to place a sample.

The connecting cap 4 is detachably connected to both the donor cell 8 and the receptor cell 1, preferably by threaded connection. Specifically, a lower circumferential surface of the connecting cap 4 is provided with an external thread, and an upper circumferential surface of the receptor cell 1 is provided with a matching internal thread. An upper circumferential surface of the connecting cap 4 is provided with an internal thread, and a lower circumferential surface of the donor cell 8 is provided with a matching external thread.

The connecting cap 4 generally has a shape of a groove 401 and is hollow in the middle, with an annular groove 402 on the lower circumferential surface, and two vent holes are symmetrically distributed on the top, preferably screw holes 403.

The screw holes 403 communicate the receptor cell 1 with ambient air and are used to discharge gas from the receptor cell 1, so that the human body fluid can fill the receptor cell 1 and tightly contact the diffusion membrane 5. In addition, the screw holes 403 can serve as sampling ports for extracting the human body fluid from the receptor cell 1.

The diffusion membrane 5 is laid flat in the groove 401 of the connecting cap 4, and the diffusion membrane 5 is fastened by threaded engagement between the connecting cap 4 and the donor cell 8. Upper and lower surfaces of the diffusion membrane 5 tightly contact the sample in the donor cell 8 and the human body fluid in the receptor cell 1, respectively. The diffusion membrane 5 is made of a dialysis membrane or skin membrane. The diffusion membrane 5 allows the sample to contact the human body fluid only through a membrane surface, implementing unidirectional migration of a chemical from the sample into the human body fluid. In addition, a position of the diffusion membrane 5 can be fastened by threaded connection between the connecting cap 4 and the donor cell 8. What's more, only the receptor cell 1 and the connecting cap 4 need to be unscrewed to disassemble the device, which facilitates replacement of the adsorbent phase 2 in the receptor cell 1.

The small sealing ring 6 can be stacked on the diffusion membrane 5, and the donor cell 8 is pressed tightly against the small sealing ring 6. The large sealing ring 3 can be embedded in the annular groove 402 of the connecting cap 4. Both the small sealing ring 6 and the large sealing ring 3 can be configured to enhance sealing performance of the device and prevent leakage at the connecting cap 4, the donor cell 8, and the receptor cell 1.

The adsorbent phase 2 can be placed inside the receptor cell 1 and is made of XAD resin, polydimethylsiloxane, or polyethylene. The adsorbent phase 2 has an enrichment effect on a target chemical and can increase a detection amount of the target chemical.

Sealing components are configured to block the screw holes 403, and are preferably the screws 7. The screws 7 can cut off connection between the receptor cell 1 and external atmosphere, ensuring the airtightness of the device, resisting environmental background interference, and preventing reduction of the human body fluid due to evaporation.

The top cover 9 covers the top of the donor cell 8. After assembly, the device is fully enclosed, preventing reduction of the human body fluid in the receptor cell 1 due to leakage or evaporation, and resisting the environmental background interference.

Preferably, the receptor cell 1, the connecting cap 4, the screws 7, the donor cell 8, and the top cover 9 are made of stainless steel, glass, or plastic.

A sampler is operated according to the following steps:

(1) assembling a transdermal migration device, including a receptor cell 1, an adsorbent phase 2, a large sealing ring 3, a connecting cap 4, a diffusion membrane 5, a small sealing ring 6, screws 7, a donor cell 8, and a top cover 9, where the receptor cell 1 is configured to hold human body fluid, the connecting cap 4 is configured to connect the receptor cell 1 and the donor cell 8, and the donor cell 8 is configured to place a sample;

(2) filling the receptor cell 1 with the human body fluid and block screw holes 403 with the screws 7; and placing the sample in the donor cell 8, which is laid flat on the diffusion membrane 5;

(3) placing the device in a 37°C incubator to conduct transdermal migration test; and

(4) periodically replacing an adsorbent and the human body fluid, and separately processing the adsorbent and the human body fluid for detection and analysis using instruments such as gas chromatography-mass spectrometry.

The above embodiments are preferred implementations of the present invention, but the implementations of the present invention are not limited to these embodiments. Any modifications, substitutions, combinations, or simplifications made without departing from the spirit and principles of the present invention should be considered equivalent alternatives and are included within the scope of protection of the present invention.

What is claimed is:

1. A transdermal migration device, comprising a receptor cell for holding human body fluid, an adsorbent phase, a connecting cap, a diffusion membrane, sealing components, a donor cell for placing a sample, and a top cover, wherein
the adsorbent phase is placed inside the receptor cell;
the connecting cap has a shape of a groove and is hollow in the middle, with upper and lower ends detachably connected to the donor cell and the receptor cell, respectively;
the diffusion membrane is laid flat in the groove of the connecting cap and fastened by the donor cell, with upper and lower surfaces of the membrane tightly contacting the sample in the donor cell and the human body fluid in the receptor cell, respectively;
the connecting cap is provided with vent holes to communicate the receptor cell with ambient air, and the sealing components are detachably plugged into the vent holes;
the top cover covers the top of the donor cell; and
the connecting cap is threadedly connected to both the donor cell and the receptor cell, and the diffusion membrane is fastened by threaded engagement between the connecting cap and the donor cell.

2. The transdermal migration device according to claim 1, wherein a lower circumferential surface of the connecting cap is provided with an external thread, and an upper circumferential surface of the receptor cell is provided with a matching internal thread; and an upper circumferential surface of the connecting cap is provided with an internal thread, and a lower circumferential surface of the donor cell is provided with a matching external thread.

3. The transdermal migration device according to claim 1, wherein a small sealing ring is provided on the diffusion membrane, and the donor cell is pressed tightly against the small sealing ring.

4. The transdermal migration device according to claim 1, wherein a large sealing ring is provided at a connection between the connecting cap and the receptor cell.

5. The transdermal migration device according to claim 4, wherein an annular groove is provided on the lower circumferential surface of the connecting cap, and the large sealing ring is fitted in the annular groove.

6. The transdermal migration device according to claim 1, wherein the vent holes are screw holes, the sealing components are screws, and the screws are threadedly connected to the screw holes.

7. The transdermal migration device according to claim 1, wherein the adsorbent phase is made of XAD resin, polydimethylsiloxane, or polyethylene.

8. The transdermal migration device according to claim 1, wherein the diffusion membrane is made of dialysis membrane or skin membrane.

9. The transdermal migration device according to claim 1, wherein the receptor cell, the connecting cap, the donor cell, and the top cover are made of stainless steel, glass, or plastic.
